(19) United States
(12) Patent Application Publication
(10) Pub. No.: US 2002/0086070 A1
(43) Pub. Date: Jul. 4, 2002

(54) Anti-inflammatory and connective tissue repair formulations

(76) Inventor: Eric Hauser Kuhrts, Bodega, CA (US)

Correspondence Address: WILSON SONSINI GOODRICH & ROSATI, 650 PAGE MILL ROAD, PALO ALTO, CA, 943041050

(21) Appl. No.: 09/982,381

(22) Filed: Oct. 17, 2001

Related U.S. Application Data
(63) Continuation-in-part of application No. 09/524,416, filed on Mar. 11, 2000.

(51) Int. Cl. A61K 35/78

(52) U.S. Cl. 424/773; 424/778

(57) ABSTRACT

Disclosed is a pharmaceutical composition including a therapeutic quantity of an a joint restorative compound selected from aminosugars, chondroitin, collagen 2, or methyl sulfonyl methane; and a therapeutic quantity of a COX-2 inhibitor having an IC50-WHMA COX-2/COX-1 ratio ranging from about 0.23 to about 3.33. Also disclosed are methods for the treatment, regeneration, and repair of connective tissue in mammals and methods for treating osteoarthritis, rheumatoid arthritis or acute pain utilizing the disclosed

### RELATED APPLICATIONS

This is a continuation-in-part of serial number 09/524,416, filed Mar. 11, 2000, which is hereby incorporated by reference as if reproduced fully herein.

### FIELD OF THE INVENTION

This invention relates to therapeutic compositions that exhibit anti-inflammatory and joint repair properties. The compositions are useful for treating osteoarthritis and rheumatoid arthritis, as well as connective tissue damaged by trauma or injury.

### BACKGROUND OF THE INVENTION

Osteoarthritis is a degenerative joint disease and is the most common form of arthritis, affecting over 20 million people in America alone, most of which are 45 years old or older. Osteoarthritis causes the cartilage that covers the bone ends to deteriorate, causing pain, inflammation, and disability. Rheumatoid arthritis affects fewer people than osteoarthritis, nonetheless rheumatoid arthritis still affects just over 2 million people in the United States alone. There are also a large number of people who suffer from problems with connective tissue damaged by trauma or injury.

There are various patents related to the use of certain joint restorative compounds for treating osteoarthritis, rheumatoid arthritis, or connective tissue damaged by trauma or injury. For instance, U.S. Pat. No. 5,364,845, 5,587,363, and 5,679,344 disclose glucosamine salts for the treatment of joint and cartilage repair. Glucosamine is an amino sugar that has a beneficial effect on cartilage metabolism. Additional benefits include protection from joint degradation and stimulating the synthesis of proteoglycans. Since articular cartilage contains proteoglycans, their stimulation results in enhanced healing of damage associated with arthritis and joint injury. Other joint restorative compounds include chondroitin, collagen 2, and methyl sulfonyl methane.

While joint resotative compounds are beneficial healing substances, they do not act in an anti-inflammatory fashion. Furthermore, patients must take most joint resotative compounds for some time, on occasion at least six weeks, before they experience some relief from joint pain. There is a real need for a faster onset of action for the quick relief of pain. Joint inflammation and pain such as that associated with osteoarthritis is the result of increased levels of pro-inflammatory prostaglandins that are derived from arachidonic acid via the enzyme cyclooxygenase. There are two types of this enzyme, COX-1 and COX-2. Non-steroidal anti-inflammatory drugs such as aspirin and ibuprofen reduce the pain and swelling of arthritis by inhibiting the COX-1 form of the enzyme, but have the side effect of causing gastric erosion if used on a regular basis. The newer arthritis drugs such as rofecoxib, and celecoxib, inhibit the COX-2 form of the enzyme, and reduce pain without causing a high incidence of gastric erosion.

The GI upset and stomach irritation caused by high doses of COX-1 inhibitors is due to their action on prostaglandin production in a manner similar to that of aspirin and aspirin-like anti-inflammatory agents. Numerous studies have shown that the relative incidence of these GI side effects can be correlated to the relative COX-2 specificity of these agents. The higher the specificity for COX-2 over COX-1, the lower the incidence of GI upsets. For instance, aspirin, with a COX-2 specificity of only 0.6, produces a greater incidence of GI distress than most botanical COX inhibitors, with a reported COX-2 specificity of nearly 4 times higher. Accordingly, cyclooxygenase inhibiting agents with increased COX-2 specificity may provide in anti-inflammatory compositions having less incidences of gastrointestinal distress or side effects.

However, too much selectivity for COX-2 over COX-1 may not be desirable. Certain side-effects may result from COX inhibitors that are extremely selective for COX-2. For example, the cardiovascular benefit of aspirin, a predominantly COX-1 non-steroidal anti-inflammatory drug (NSAID), is thought to be due to its activity as an anti-platelet aggregating drug. COX-2 inhibition does not result in anti-platelet aggregation. Current pharmaceutical COX-2 inhibitors, such as celecoxib or rofecoxib, are highly specific COX-2 inhibitors, and would not be expected to have any COX-1 inhibitory activity. Thus, the cardiac-related side effects that have been noted with the use of some COX-2 specific inhibitors may be related to the lack of any COX-1 inhibition while significantly inhibiting COX-2.

What is needed are compositions and methods that address the problems noted above.

### SUMMARY OF THE INVENTION

In an aspect, the invention relates to a pharmaceutical composition comprising a therapeutic quantity of an a joint restorative compound selected from amino sugars, chondroitin, collagen 2, or methyl sulfonyl methane; and a therapeutic quantity of a COX-2 inhibitor having an IC50-WHMA COX-2/COX-1 ratio ranging from about 0.23 to about 3.33.

### DETAILED DESCRIPTION OF THE INVENTION

The inventors have unexpectedly discovered that the above noted problems can be solved by a pharmaceutical composition comprising a therapeutic quantity of an a joint restorative compound selected from aminosugars, chondroitin, collagen 2, or methyl sulfonyl methane; and a therapeutic quantity of a COX-2 inhibitor having an IC50-WHMA COX-2/COX-1 ratio ranging from about 0.23 to about 3.33. COX-2 inhibitors having an IC50-WHMA COX-2/COX-1 ratio more than about 3.33 may exhibit undesirable cardiological side effects.

Joint restorative compounds useful in the practice of this invention comprise aminosugars, chondroitin, collagen, collagen 2, and methyl sulfonyl methane. In a preferred embodiment, the aminosugars according to the invention comprise glucosamine salts, most preferably glucosamine sulfate. In a preferred embodiment, the chondroitin comprises chondroitin sulfate. The collagen or collagen 2 according to the invention may be obtained from chicken cartilage, or shark cartilage or similar sources thereto.

Preferable doses of joint restorative compounds in the inventive composition range from about 150 mg to about 1500 mg., more preferably ranging from about 250 mg. to about 1000 mg of the joint restorative compound.

The COX-2 inhibitors useful in the practice of this invention (the “recited COX-2 inhibitors”) may be obtained from a variety of sources, so long as the recited COX-2 inhibitor has an IC50-WHMA COX-2/COX-1 ratio ranging from about 0.23 to about 3.33. This may be obtained, for example, by mixing together two or more COX-2 inhibitors so as to arrive at an average IC50-WHMA COX-2/COX-1 ratio in the range from about 0.23 to about 3.33.

Preferably, the benefits of the invention may accrue if the recited COX-2 inhibitor is a botanical COX-2 inhibitor. In a especially preferred embodiment, the botanical COX-2 inhibitor comprises hops (Humulus lupus L) or Polygonum Cuspidatum (a member of the buckwheat family commonly known as japanese knotweed).

Hops has been in use by the beer industry for hundreds of years. More recently, hops has been shown to exhibit estrogenic activity (J Agric Food Chem 2001, 2001 May, 49(5): 2472-2479), and other metabolic and endocrine effects. The estrogenic property of hops is believed to be due to the presence of the flavonoid, 8-prenylnaringenin, which is present in some beers in small quantities. There are however, at least six flavonoids that can be isolated from hops, and some of these flavonoids have antiproliferative and cytotoxic effects. The phytoestrogens in hops have also been shown to inhibit growth of human breast cancer cells. The unique flavonoid compounds isolated from hops therefore have potential as cancer chemopreventative agents by effecting the metabolism of carcinogens. Hops also exhibits antimicrobial properties.

The anti-inflammatory properties of hops extract has been traced to one of the bitter principles or resins in hops called humulon. In one study, humulon inhibited arachidonic acid-induced inflammatory ear edema in mice (Yasukawa, K et al, Oncology 1995, Mar; 52(2): 156-158), and also inhibited skin tumor formation following initiation with a chemical challenge. Humulon, the alpha acid contained in hops, has also been shown to suppress cyclooxygenase-2 induction at the level of transcription (Yamamoto K, et al, FEBS Lett 2000 Jan 14, 465(2-3: 103-106). Humulon, therefor, could be considered a COX-2 inhibitor. Furthermore, humulon suppressed the TNFalpha-dependent cyclooxygenase-2 induction with an IC(50) of about 30 nM, a fairly low concentration.

Hops according to the invention may be used in its entirety, as whole hops powder for instance, or may be used as extracts of the hops flowers, pure humulon or other active principles isolated from hops. Extraction of hops also yields various essential oils, oleoresins, and alpha and beta acids. The primary beta acids in hops are lupulone, colupulone, and adlupulone. Hops resin is obtained from the yellow vesicles in the flowers of the hops plant. Extraction of hops resin is usually done using accepted extraction techniques with such solvents as hexane or ethyl alcohol, which concentrates the alpha and beta acids.

A more preferred extraction technique is using liquid carbon dioxide under supercritical conditions can be used to separate the alpha and beta fractions. Supercritical fluid technology is a more recent and superior means of extracting and concentrating the active principles that are contained in botanical extracts. Furthermore, supercritical fluid extraction is not a solvent based system, so it results in solvent free extractions, and is less harmful to the environment, because there is no need to evaporate toxic organic solvents. CO2 is the most commonly used material in supercritical fluid extraction and fractionation. Supercritical CO2 extraction also allows for better separation and fractionation of certain components in hops that may not be necessary for a particular application, such as the elimination of estrogenic components which may not be needed in an anti-inflammatory formula. For instance, ethanol extracts of hops are known undesirably to possess strong estrogenic properties.

Polygonum Cuspidatum is a member of the buckwheat family (polygonaceae), commonly known as japanese knotweed. This plant is a native of eastern Asia, but also grows wild throughout northeastern America and southern Canada. The roots Polygonum Cuspidatum contain a large amount of resveratrol, a stilbene which is a powerful anti-oxidant, and exhibits anti-inflammatory, anti-mutagen, and anti-carcinogenic properties. Resveratrol also inhibits blood platelet aggregation, making it a beneficial cardiovascular compound. Recently, resveratrol was found to inhibit COX-2 by dose dependently reducing prostaglandin E-2 (PGE2) production in human mammary epithelial cells. The dried roots of Polygonum Cuspidatum contain about 5-8% resveratrol. By using various extracting techniques to concentrate the amount of resveratrol in Polygonum Cuspidatum, high yield powders have been obtained that contain up to 20% resveratrol. Therefore, 100 mg. of Polygonum Cuspidatum extract may deliver 20 mg. of actual resveratrol.

Other plant sources of resveratrol include grapes or wine (Vitus vinfera), which contains 1-13 mg. of resveratrol per liter, with an average of about 5 mg./liter. Resveratrol is also present in the following plants; Polygonum multiflorum, Pterolobium hexapetallum, Cassia garrettiana Carib, Cassia quinquangulata, Arachis hypogaea, Eucalyptus globulus, and Bauhinia racemosa Lamk, Veratrum grandiflorum, and Veratrum formosanum.

While resveratrol is perhaps the most widely studied of the constituents in Polygonum cuspidatum, there are also other active substances contained therein, such as emodin, polydatin, and piceid. These may contribute to the beneficial effects of the plant extract in a synergistic fashion, but also exhibit some of the same and other pharmacological properties as resveratrol.

In-vitro testing or screening of the recited COX-2 inhibitors can be conducted by measuring the inhibition of prostaglandin E-2, a pro-inflammatory prostaglandin. This results in the calculation of the IC50 values, or the amount or concentration of the compound needed to inhibit COX-2 by 50%. This model measures the production of prostaglandin E2 (PGE2) by the COX-2 enzyme related pathways, when stimulated by LPS. Such assays are now considered to represent a more complete in-vitro picture of COX-2/COX-1 selectivity and potency. To determine the COX-2/COX-1 inhibitory activity according to the invention the William Harvey Modified Human Whole Blood /Cell Assay (WHMA) is used, as set forth in T. D. Warner et al., Nonsteroid drug selectivities for cyclo-oxygenase-1 rather than cyclo-oxygenase-2 are associated with human gastrointestinal toxicity: A full in vitro analysis, Proc. Natl. Sci. USA 96:7563-68 (1999), hereby incorporated by reference in its entirety. The results from this assay are used to calculate the IC50-WHMA COX-2/COX-1 ratio, which is simply the numerical ratio of the COX-2 IC50 divided by the COX-1 IC50 ratio, obtained using the WHMA.

Preferable doses of the recited COX-2 inhibitor range from about 50 mg. to about 1000 mg of the recited COX-2 inhibitor in the inventive compositions.

Dosage forms comprising according to the invention may be taken numerous times during the day or may be incorporated into sustained-release formulations to enable a single daily or nightly dose. Such sustained-release formulations provide for more effective suppression of pro-inflammatory prostaglandins due to cumulative inhibition. In addition, sustained-release formulations provide long lasting pain relief. Useful dosage forms include without limitation oral forms such as tablets, capsules, beads, granules, aggregates, powders, gels, solids, semi-solids, and suspensions. Lotions, transdermal delivery systems, including dermal patches, aerosols or nasal mists, suppositories, salves and ointments are also useful.

A variety of additives can be incorporated into the inventive compositions for their intended functions. These additives are usually used in small amounts.

Useful additives include, for example, gelatin, vegetable proteins such as sunflower protein, soybean proteins, cotton seed proteins, peanut proteins, rape seed proteins, blood proteins, egg proteins, acrylated proteins; water-soluble polysaccharides such as alginates, carrageenans, guar gum, agar-agar, gum arabic, and related gums (gum ghatti, gum karaya, gum tragacanth), pectin; water-soluble derivatives of cellulose: alkylcelluloses, hydroxyalkylcelluloses and hydroxyalkylalkylcelluloses, such as methylcellulose, hydroxymethylcellulose, hydroxyethylcellulose, hydroxypropylcellulose, hydroxyethylmethylcellulose, hydroxpropylmethylcellulose, hydroxbutylmethylcellulose, cellulose esters and hydroxyalkylcellulose esters such as: cellulose acetate phthalate (CAP), carboxyalky I celluloses, carboxyalkylalkylcelluloses, carboxyalkylcellulose esters such as carboxymethyl cellulose and their alkali metal salts; water-soluble synthetic polymers such as polyacrylic acids and polyacrylic acid esters, polymethacrylic acids and polymethacrylic acid esters, polyvinylacetates, polyvinylalcohols, polyvinylacetatephthalates (PVAP), polyvinylpyrrolidone (PVP), PVP/vinyl acetate copolymer, and polycrotonic acids; also suitable are phthalated gelatin, gelatin succinate, crosslinked gelatin, shellac, water-soluble chemical derivatives of starch, cationically modified acrylates and methacrylates possessing, for example, a tertiary or quaternary amino group, such as the diethylan-finoethyl group, which may be quaternized if desired; and other similar polymers.

Processing aids such as sucrose, polydextrose, maltodextrin, lactose, maltose, stearic acid, microcrystalline cellulose, and the like may also be used. Examples of classes of additives include excipients, lubricants, oils, hydrocolloid suspending agents, buffering agents, disintegrating agents, stabilizers, foaming agents, pigments, coloring agents, fillers, bulking agents, sweetening agents, flavoring agents, fragrances, release modifiers, etc.

An useful composition according to the invention is a sustained-release composition comprising a sustained-release form of the recited COX-2 inhibitor, and a joint restorative compound in either immediate-release or sustained-release form. By providing the recited COX-2 inhibitor in sustained-release form, more effective inhibition of the cyclooxegenase enzyme is possible due to the accumulative manner in which the enzyme is inhibited. This will also prolong the duration of action for the active principles in the rectited COX-2 inhibitor. By providing a slow but constant release of active principles, levels of pro-inflammatory prostaglandin E-2 are kept reduced, thereby providing for long lasting pain relief, throughout the day or at night while asleep.

Sustained release within the scope of this invention can be taken to mean any one of a number of extended release dosage forms. The following terms may be considered to be substantially equivalent to sustained release, for the purposes of the present invention: continuous release, sustained release, delayed release, depot, gradual release, long-term release, programmed release, prolonged release, proportionate release, protracted release, repository, retard, slow release, spaced release, sustained release, time coat, timed release, delayed action, extended action, layered-time action, long acting, prolonged action, repeated action, slowing acting, sustained action, sustained-action medications, and extended release. Further discussions of these terms may be found in Lesczek Krowczynski, Extended-Release Dosage Forms, 1987 (CRC Press, Inc.), hereby incoporated by reference.

The various sustained release technologies cover a very broad spectrum of drug dosage forms. Sustained release technologies include, but are not limited to physical systems and chemical systems. Physical systems include, but are not limited to, reservoir systems with rate-controlling membranes; microencapsulation; macroencapsulation; membrane systems; reservoir systems without rate-controlling membranes such as hollow fibers, ultra microporous cellulose triacetate, or porous polymeric substrates and foams; monolithic systems, including those systems physically dissolved in non-porous, polymeric, or elastomeric matrices (e.g., non-erodable, erodable, environmental agent ingression, and degradable); and materials physically dispersed in non-porous, polymeric, or elastomeric matrices (e.g., non-erodable, erodable, environmental agent ingression, and degradable); laminated structures, including reservoir layers chemically similar or dissimilar to outer control layers; and other physical methods, such as osmotic pumps or adsorption onto ion-exchange resins.

Chemical systems include, but are not limited to, chemical erosion of polymer matrices (e.g., heterogeneous, or homogeneous erosion), or biological erosion of a polymer matrix (e.g., heterogeneous, or homogeneous).

Hydrogels may also be employed as described in “Controlled Release Systems: Fabrication Technology”, Vol. II, Chapter 3; p 41-60; “Gels For Drug Delivery”, Edited By Hsieh, D., incorporated by reference.

Sustained release drug delivery systems may also be categorized under their basic technology areas, including, but not limited to, rate-preprogrammed drug delivery systems, activation-modulated drug delivery systems, feedback-regulated drug delivery systems, and site-targeting drug delivery systems.

Furthermore, compositions according to the invention may be administered or coadministered with conventional pharmaceutical binders, excipients and additives. Many of these are sustained-release polymers which can be used in sufficient quantities to produce a sustained-release effect. These include, but are not limited to, gelatin, natural sugars such as raw sugar or lactose, lecithin, mucilage, plant gums, pectin's or pectin derivatives, algal polysaccharides, glucomannan, agar and lignin, guar gum, locust bean gum, acacia gum, xanthan gum, carrageenan gum, karaya gum, tragacanth gum, ghatti gum, starches (for example corn starch or amylose), dextran, polyvinyl pyrrolidone, polyvinyl acetate, gum arabic, alginic acid, tylose, talcum, lycopodium, silica gel (for example colloidal), cellulose and cellulose derivatives (for example cellulose ethers, cellulose ethers in which the cellulose hydroxyl groups are partially etherified with lower saturated aliphatic alcohols and/or lower saturated, aliphatic oxyalcohols, for example methyl oxypropyl cellulose, methyl cellulose, hydroxypropyl methyl cellulose, hydroxypropyl methyl cellulose phthalate, cross-linked sodium carboxymethylcellulose, cross-linked hydroxypropylcellulose, high-molecular weight hydroxymethylpropycellulose, carboxymethyl-cellulose, low-molecular weight hydroxypropylmethylcellulose medium-viscosity hydroxypropylmethylcellulose hydroxyethylcellulose, hydroxypropylcellulose, hydroxypropylmethylcellulose, sodium carboxymethylcellulose, alkylcelluloses, ethyl cellulose, cellulose acetate, cellulose propionate (lower, medium or higher molecular weight), cellulose acetate propionate, cellulose acetate butyrate, cellulose triacetate, methyl cellulose, hydroxypropyl cellulose, or hydroxypropylmethyl cellulose), fatty acids as well as magnesium, calcium or aluminum salts of fatty acids with 12 to 22 carbon atoms, in particular saturated (for example stearates such as magnesium stearate), polycarboxylic acids, emulsifiers, oils and fats, in particular vegetable (for example, peanut oil, castor oil, olive oil, sesame oil, cottonseed oil, corn oil, wheat germ oil, sunflower seed oil, cod liver oil, or high melting point hydrogenated vegetable oil such as can be produced from soy beans); glycerol esters and polyglycerol esters of saturated fatty acids C12H24O2 to C18H36O2 and their mixtures, it being possible for the glycerol hydroxyl groups to be totally or also only partly esterified (for example mono-, di- and triglycerides); pharmaceutically acceptable mono- or multivalent alcohols and polyglycols such as polyethylene glycol and derivatives thereof, esters of aliphatic saturated or unsaturated fatty acids (2 to 22 carbon atoms, in particular 10-18 carbon atoms) with monovalent aliphatic alcohols (1 to 20 carbon atoms) or multivalent alcohols such as glycols, glycerol, diethylene glycol, pentacrythritol, sorbitol, mannitol and the like, which may optionally also be etherified, esters of citric acid with primary alcohols, acetic acid, urea, benzyl benzoate, dioxolanes, glyceroformals, tetrahydrofurfuryl alcohol, polyglycol ethers with C1-C12-alcohols, dimethylacetamide, lactamides, lactates, ethylcarbonates, silicones (in particular medium-viscous polydimethyl siloxanes), calcium carbonate, sodium carbonate, calcium phosphate, sodium phosphate, magnesium carbonate and the like.

Other substances that may be used include: cross-linked polyvinyl pyrrolidone, carboxymethylamide, potassium methacrylatedivinylbenzene copolymer, high-molecular weight polyvinylacohols, low-molecular weight polyvinylalcohols, medium-viscosity polyvinylalcohols, polyoxyethyleneglycols, non-cross linked polyvinylpyrrolidone, polyethylene glycol, sodium alginate, galactomannone, carboxypolymethylene, sodium carboxymethyl starch, sodium carboxymethyl cellulose or microcrystalline cellulose; polymerizates as well as copolymerizates of acrylic acid and/or methacrylic acid and/or their esters, such as, but not limited to poly(methyl methacrylate), poly(ethyl methacrylate), poly(butyl methacylate), poly (isobutyl methacrylate), poly(hexyl methacrylate), poly (isodecyl methacrylate), poly(lauryl methacrylate), poly(phenyl methacrylate), poly(methyl acrylate), poly(isopropyl acrylate), poly(isobutyl acrylate), or poly(octadecyl acrylate); copolymerizates of acrylic and methacrylic acid esters with a lower ammonium group content (for example Eudragit® RS, available from Rohm, Somerset, N.J.), copolymerizates of acrylic and methacrylic acid esters and trimethyl ammonium methacrylate (for example Eudragit® RL, available from Rohm, Somerset, N.J.); polyvinyl acetate; fats, oils, waxes, fatty alcohols; hydroxypropyl methyl cellulose phthalate or acetate succinate; cellulose acetate phthalate, starch acetate phthalate as well as polyvinyl acetate phthalate, carboxy methyl cellulose; methyl cellulose phthalate, methyl cellulose succinate, -phthalate succinate as well as methyl cellulose phthalic acid half ester; zein; ethyl cellulose as well as ethyl cellulose succinate; shellac, gluten; ethylcarboxyethyl cellulose; ethylacrylate-maleic acid anhydride copolymer; maleic acid anhydride-vinyl methyl ether copolymer; styrol-maleic acid copolymerizate; 2-ethyl-hexyl-acrylate maleic acid anhydride; crotonic acid-vinyl acetate copolymer; glutaminic acid/glutamic acid ester copolymer; carboxymethylethylcellulose glycerol monooctanoate; cellulose acetate succinate; polyarginine; poly (ethylene), poly (ethylene) low density, poly (ethylene) high density, poly (propylene), poly (ethylene oxide), poly (ethylene terephthalate), poly (vinyl isobutyl ether), poly (vinyl chloride) or polyurethane. Mixtures of any of the substances or materials listed herein may also be used in the practice of the invention.

The compositions according to the invention may be orally administered or coadministered in a liquid dosage form such as a tea or soft drink. For the preparation of solutions or suspensions it is, for example, possible to use water or physiologically acceptable organic solvents, such as alcohols (ethanol, propanol, isopropanol, 1,2-propylene glycol, polyglycols and their derivatives, fatty alcohols, partial esters of glycerol), oils (for example peanut oil, olive oil, sesame oil, almond oil, sunflower oil, soya bean oil, castor oil, bovine hoof oil), paraffins, dimethyl sulphoxide, triglycerides and the like.

In the case of drinkable solutions the following substances may be used as stabilizers or solubilizers: lower aliphatic mono- and multivalent alcohols with 2-4 carbon atoms, such as ethanol, n-propanol, glycerol, polyethylene glycols with molecular weights between 200-600 (for example 1 to 40% aqueous solution), gum acacia or other suspension agents selected from the hydrocolloids may also be used.

It is also possible to add preservatives, stabilizers, buffer substances, flavor correcting agents, sweeteners, colorants, antioxidants and complex formers and the like. Complex formers which may be for example be considered are: chelate formers such as ethylene diamine retrascetic acid, nitrilotriacetic acid, diethylene triamine pentacetic acid and their salts.

Furthermore, sustained release compositions according to the invention may be administered separately, or may co-administered with other inventive sustained release or immediate-release biological equivalents or other therapeutic agents. Co-administration in the context of this invention is defined to mean the administration of more than one therapeutic in the course of a coordinated treatment to achieve an improved clinical outcome. Such co-administration may also be coextensive, that is, occurring during overlapping periods of time.

The pharmaceutical compositions of the present invention may be used to treat, regenerate, and repair connective tissue in mammals; and may also be used to treat osteoarthritis, rheumatoid arthritis or acute pain. Dosing is by conventional means for the dosage selected. Conventional methods (such as dose ranging studies) may be used to determine dosage amounts; alternatively preferable dosage ranges have been disclosed elsewhere herein.

An advantage of the invention is that the combination of an amino sugar with a recited COX-2 inhibitor can result in a synergistic increase in the analgesic activity of the composition. The mechanism by which this effect occurs is not certain, but may involve altered COX-2 inhibitor metabolism/pharmacokinetics, resulting in effective pain relief at a lower dose. For instance, the synergistic effect may increase the maximum concentration of the recited COX-2 inhibitor in the blood or blood plasma, or may prolong or enhance the bioavailability of the recited COX-2 inhibitor or its metabolites, or may impact other pathways that directly or indirectly interact with the pathways involving cyclooxygenase-2. In an embodiment, the combination of a glucosamine salt with a hops extract could result in a significantly increased analgesic effect from the hops component. Such a synergistic increase in the analgesic activity would be useful for inventive compositions for and methods of treating joint pain or other types of pain, including acute pain, or pain due to trauma or injury, or for improved inhibition of cyclooxygenase-2 in mammals.

An advantage of the invention is that it provides an anti-inflammatory and pain relieving effect while reducing the danger of gastric erosion from frequent usage, such as would be encountered with a composition that did not comprise a recited COX-2 inhibitor. Still another benefit is the fast onset of pain relief action due to the immediate anti-inflammatory effects of the recited COX-2 inhibitor, which may operate cooperatively with the restorative properties of the joint restorative compound.

Surprisingly, by combining a joint restorative compound with a recited COX-2 inhibitor, significantly more effective joint pain relief is achieved initially, with continued improvement over time as the joint restorative compound begins to work its way into cartilage metabolism. Additionally, the combination of a joint restorative compound with the recited COX-2 inhibitor also results in more effective reduction of pain than either the joint restorative compound or the recited COX-2 inhibitor alone. This may translate into a reduction in dose amount, or an increase in the analgesic efficacy of the inventive pharmaceutical composition. Therefore, the inventive pharmaceutical composition may result in significantly greater analgesic effects than either ingredient alone.

While the present invention is described above in connection with the preferred or illustrative embodiments, those embodiments are not intended to be exhaustive or limiting of the invention, but rather, the invention is intended to cover any alternatives, modifications, or equivalents that may be included within its scope as defined by the appended claims.

### EXAMPLES

### Example 1

Glucosamine sulfate is blended with a hops extract powder that is a blend of immediate release and sustained-release supercritical CO2 extract and a lubricant in the following amounts:

| | |
|---|---|
| Glucosamine sulfate | 500 mg. |
| Hops extract (42% humulon) | 250 mg. |
| Magnesium stearate | 3.5 mg. |

The resulting composition is dissolved in dimethylsulfoxide and is tested according to the William Harvey Modified Human Whole Blood/Cell Assay, as set forth in T. D. Warner et al., Nonsteroid drug selectivities for cyclo-oxygenase-1 rather than cvclo-oxygenase-2 are associated with human gastrointestinal toxicity: A full in vitro analysis, Proc. Natl. Sci. USA 96:7563-68 (1999).

Human whole blood (8 concentrations, n=4) is collected of blood by venapuncture into heparin. For determining COX-1: Incubation of test compound(s) for 1 hour, with addition of stimulus (A23187) for 30 minutes. For COX-2: Incubation of test compounds for 1 hour, addition of stimulus (A23187) for 30 minutes. Following this, measure TxB2 by RIA (index of COX-1 activity); measure PGE2 by RIA (index of COX-2 activity). The results are expressed as % control, and COX-2/COX-1 ratio is calculated.

### Example 2

Glucosamine sulfate is blended with a hops extract powder that is a blend of immediate release and sustained-release supercritical CO2 extract and a lubricant and filled into two piece hard shell capsules according to the following formula;

Each capsule contains:

| | |
|---|---|
| Glucosamine sulfate | 500 mg. |
| Hops extract (42% humulon) | 250 mg. |
| Magnesium stearate | 3.5 mg. |

The resulting composition is dissolved in dimethylsulfoxide and its effect on cyclooxigenase 2 (COX-2) activity is measured using the 184B5/HER cell line as described by Zhai et. al. in Cancer Research, (1993), 53, 2272-2278. In this assay, if basal COX-2 activity is inhibited, production of prostaglandin E-2 (PGE2) is significantly reduced because the synthesis of PGE2 from arachidonic acid (sodium arachidonate is added to the medium) is blocked or reduced by the hops extract. PGE2 production released by cells can be measured by enzyme immunoassay (ELISA) and shown to be significantly reduced.

As an additional test, the above formulation can be used to determine inhibition of recombinant human COX-2 enzyme activity. In that model, radioactive arachidonic acid is added to a reaction mixture containing human recombinant COX-2 enzyme and other chemicals. Levels of prostaglandin E-2 are measured using high pressure liquid chromatography (HPLC). The percent activity is determined by comparing levels of synthesis of PGE2 in control incubations with levels seen in incubation mixtures containing known concentrations of test compounds.

### Example 3

A sustained-release tablet formulation. All of the ingredients are first blended and then subjected to direct compression in a tablet press according to the following formula;

Each tablet contains:

| | |
|---|---|
| Glucosamine sulfate | 500 mg. |
| Hops extract (sustained-release powder) | 500 mg. |
| Stearic acid | 50 mg. |
| Citrus Pectin | 20 mg. |
| Di-calcium phosphate | 20 mg. |
| Micro-crystalline cellulose | 20 mg. |
| Magnesium stearate | 5 mg. |

The sustained-release hops extract used in the above example is microencapsulated into a direct compression powder with a high yield after conversion from a supercritical CO2 paste. The resulting tablet provides sustained-release of the active ingredients over a 6-8 hour time period, but with a loading dose that is released over the first hour to provide initial quick pain relief as well as long lasting relief with continued use.

### Example 4

The 3.5 kilos of glucosamine HCL and 0.5 kilos of hops extract, were charged to a high shear mixer with a hot water jacket to allow circulating hot water to keep the vessel hot. After mixing, hydrogenated soy oil powder was added to the vessel at a 2% weight gain. The work input was increased to 2000 RPM and then adjusted down to about 600 RPM for about 3 minutes. The circulating hot water and the high shear of the mixer together melted the oil. The work input of the mixer provided energy to help melt the oil and mixed the core ingredients. The powder was discharged into a cooler mounted below the unit. The resulting particles were small, powder like, free flowing, and exhibited excellent sustained-release properties with a prolonged release profile at only 2% by weight of oil. These micro-encapsulated particles can be blended with suitable suspending agents, flavors, and sweeteners to produce a sustained-release drink mix that enables a larger dose of glucosamine to be consumed in a single daily or 24 hour dose. Alternatively, the powder can be encapsulated in two-piece hard shell capsules.

What is claimed is:

1. A pharmaceutical composition comprising
a therapeutic quantity of an a joint restorative compound selected from aminosugars, chondroitin, collagen 2, or methyl sulfonyl methane; and
a therapeutic quantity of a COX-2 inhibitor having an IC50-WHMA COX-2/COX-1 ratio ranging from about 0.23 to about 3.33.

2. The Pharmaceutical composition of claim 1, wherein the COX-2 inhibitor comprises a botanical COX-2 inhibitor.

3. The pharmaceutical composition of claim 1, wherein the amino sugar comprises glucosamine, glucosamine salts, and mixtures thereof.

4. The pharmaceutical composition of claim 1, wherein the COX-2 inhibitor comprises hops.

5. The pharmaceutical composition of claim 4, wherein the hops comprises a hops extract.

6. The pharmaceutical composition of claim 5, wherein the hops extract is obtained through supercritical carbon dioxide extraction of whole hops.

7. The therapeutic composition of claim 1, wherein the dose of the Cox-2 inhibitor ranges from about 50 mg. to about 1,000 mg.

8. The pharmaceutical composition of claim 1, wherein the dose of the joint restorative compound ranges from about 150 mg. to about 1,500 mg.

9. A method for the treatment, regeneration, and repair of connective tissue in mammals comprising:
selecting the pharmaceutical composition of claim 1; and
administering a therapeutically effective amount of the pharmaceutical 5 composition to a mammal in need thereof.

10. A method for treating osteoarthritis, rheumatoid arthritis or acute pain comprising:
selecting the pharmaceutical composition of claim 1; and
administering a therapeutically effective amount of the pharmaceutical composition in need thereof.

11. The method of claim 9, wherein the COX-2 inhibitor comprises a botanical COX-2 inhibitor.

12. The method of claim 10, wherein the COX-2 inhibitor comprises a botanical COX-2 inhibitor.

13. The method of claim 9, wherein the COX-2 inhibitor comprises hops.

14. The method of claim 10, wherein the COX-2 inhibitor comprises hops.

15. The pharmaceutical composition of claim 1, wherein the ingredients are in sustained-release or immediate-release form, or a blend of sustained-release and immediate-release.

16. The pharmaceutical composition of claim 15, wherein the sustained-release form comprises: algal polysaccharides, chitosan, pectin, glucomannan, guar gum, xanthan gum, gum arabic, gum karaya, locust bean gum, keratin, laminaran, carrageenan, cellulose, modified cellulosic substances such as cellulose ether derivatives; methylcellulose, hydroxypropylmethylcellulose, hydroxypropylcellulose, hydroxyethylcellulose, sodiumcarboxymethylcellulose, carboxymethylcellulose carboxypolymethylene, acrylic resin polymers, polyacrylic acid and homologues, polyethylene glycol, polyethylene oxide, polyhydroxylalkyl methacrylate, polyvinylpyrollidine, polyacrylamide, agar, zein, stearic acid, hydrogenated vegetable oils, carnauba wax, or gelatin.

17. The pharmaceutical composition of claim 1, wherein the pharmaceutical composition comprises an oral dosage forms that comprises tablets, capsules, beads, granules, aggregates, powders, gels, solids, semi-solids, or suspensions.

18. The pharmaceutical composition of claim 1, wherein the pharmaceutical composition comprises a topical dosage forms that comprises lotions, transdermal delivery systems, including dermal patches, aerosols, nasal mists, suppositories, salves or ointments.
